(19) United States
(12) Patent Application Publication
(10) Pub. No.: US 2011/0054030 A1
(43) Pub. Date: Mar. 3, 2011

(54) USE OF EICOSAPENTAENOIC ACID AND/OR DOCOSAHEXAHENOIC ACID IN WOMEN WITH ENDOMETRIOSIS

(75) Inventors: Felice Petraglia, Siena (IT); Stefano Luisi, Pisa (IT); Andrea Lacorte, Pisa (IT); Germano Tarantino, Pisa (IT)

(73) Assignee: PHARMANUTRA S.R.L., 56014 Pisa (IT)

(21) Appl. No.: 12/865,009

(22) PCT Filed: Jan. 29, 2009
(86) PCT No.: PCT/EP09/50997

§ 371 Date: Nov. 16, 2010

(30) Foreign Application Priority Data
Jan. 30, 2008 (IT) MI2008A000145

(51) Int. Cl. A61K 31/202; A61K 31/20; A61P 25/00

(52) U.S. Cl. 514/560; 514/558

(57) ABSTRACT

The present invention pertains to the use of essential fatty acids for the preparation of a food composition for treating in particular post-surgical pain and/or pain due to a post-surgical relapse in women having undergone an operation for endometriosis. Said essential fatty acids are preferably selected from the group consisting of long-chain polyunsaturated fatty acids of the omega-3 series, and in particular from the group comprising EPA and/or DHA.

The present invention relates to the use of essential fatty acids for the preparation of a food composition for treating endometriosis pain and, in particular, for treating postsurgical pain and/or pain due to a postsurgical relapse in women having undergone an operation for endometriosis. Said essential fatty acids are preferably selected from the group consisting of long-chain polyunsaturated fatty acids of the omega-3 series, and in particular from the group comprising eicosapentaenoic acid (EPA) and docosahexaenoic acid (DHA).

Endometriosis is a benign gynecologic pathology characterized by the presence of endometrial cells, glands and stroma outside the uterus, in the peritoneal cavity and the pelvis. Although the prevalence of endometriosis in the general population is not exactly known, it represents a frequent pathology that has been experiencing an enormous increase among women of reproductive age with a peak incidence between 30 and 50 years of age, while it only occurs as an exception during adolescence. The percentage of women with pelvic pain and/or infertiliy varies, in fact, from 20 to 90 percent. It is one of the most controversial gynecologic pathologies: Despite the growing attention it has received in the past few decades from the entire scientific community, it remains, in fact, a mystery to be solved on account of its peculiar physiopathologic implications.

Endometriosis has a multifactor etiology. Whatever its cause, endometriosis follows the dissemination and settling of endometrium in ectopic sites, or the differentiation of other types of tissue into endometrium. Successive proliferation is conditioned by the general endocrinal state and the local production of hormonal, immune, angiogenetic and growth factors. The microenvironment is, in fact, held responsible for the progression of endometriosis from superficial to profound.

In 20 to 25 percent of the cases, endometriosis is asymptomatic and is coincidentally diagnosed during a laparoscopic intervention performed in most cases due to sterility.

The main symptoms of the disease are: pelvic pain, dysmenorrhoea, dyspareunia, infertility and menometrorrhagia. The pain generally sets in a few days before menstrual flow and tends to accentuate during and especially at the end of menstruation. This is due to the increase in ectopic deposits which become more voluminous during menstrual flow due to blood accumulating therein, as well as due to inflammatory reaction and hypertrophy of the surrounding connective tissue that may be followed by peritoneal irritation with formation of scar tissue and, thus, of adhesions between pelvic and abdominal organs.

Dyspareunia is particularly accentuated during the pre- and post-menstrual period; it becomes constant in those cases where pelvic localization is associated with fixed uterine retroversion.

In some women, the main sign of endometriosis is infertility; conception may be hindered in various ways, by inflammation of the surrounding tissues and the ensuing formation of adhesions in diffuse tubo-ovarian and/or pelvic sites. In fact, webs of scar tissue may distort the pelvic anatomy; the adhesions may, in turn, interfere with the release of oocyte from the ovary or with the capability of the tube to capture the latter.

The different locations are responsible for particular clinical pictures which may, in the case of bladder or rectal locations, include urination disorders (dysuria, tenesmus) or intestinal disorders (diarrhea, constipation, rectal tenesmus). The most frequent site is the ovary; lesions may be superficial in the form of small nodules, or may go deeper into the ovarian parenchyma and present themselves in the form of veritable endometrial cysts of variable diameter, with generally regular and thick walls and a piceous content so as to be defined as “chocolate cysts”. The complication to be feared most is, however, rupture of the endometrial cyst with possible development of acute abdominal syndrome.

Not only does laparoscopy permit diagnosis in many asymptomatic cases, it also remains the diagnostic procedure of choice.

It is possible therewith to observe the pelvis and the peritoneal surface on an enlarged scale, thus also allowing possible adhesions or other endometrial lesions of small dimensions to be visualized, in addition to ovarian endometriomas. A biopsy of the cystic and/or peritoneal lesion, as well as a corresponding histological examination, permit a definitive and safe diagnosis.

It is also possible with laparoscopy to study the disease by examining the number, dimension and location of the endometrial deposits, thus establishing a score that will provide an indication of the stage of the disease (minimal, slight, moderate, serious).

The diagnostic action is generally also followed by therapeutic action in the form of surgery. The surgical treatment of endometriosis is directed at eliminating either the painful symptomatology or the sterility that may be associated with this pathology. The least invasive and least costly surgical approach is, as always, to be preferred. Laparoscopy thus proves to be the method of choice since it reduces costs, morbidity and the occurrence of post-surgical adhesions. Laparotomy, on the other hand, is to be reserved for patients with advanced-stage disease in whom preservation of the reproductive function is not indispensible. In the course of laparoscopy, the endometrial lesions can be removed using a bipolar coagulator or a laser method.

The objective of surgical treatment of endometriosis is to reestablish, wherever possible, the normal anatomic integrity and to eliminate as many endometrial deposits as possible.

Surgical success is directly correlated with the severity of endometriosis. The recurrence rate of endometriosis reported after surgery is 20% within 5 years, and whenever there is a recurrence, this particularly involves the reappearance of the typical painful symptoms of the disease.

Where the objective of surgery is to improve the symptoms and not to achieve pregnancy, the return of the symptoms is delayed by a post-surgical medical treatment that lasts little more than six months.

The pharmacological medical treatment should be reserved for patients still suffering from pain and/or dyspareunia following the surgical intervention and/or following a relapse after said intervention. The drugs most commonly used in clinical practice include: danazole, oral contraceptives, GnRH analogues (GnRH=gonadrotrophin-releasing hormone), and progestins.

Danazole has proven to be efficient in reducing the symptoms of endometriosis, yet its side effects may preclude its use. Danazole is a synthetic androgen which inhibits LH and FSH, creating a relatively hypoestrogenic state. Endometrial atrophy is the principal mechanism on which the regression of symptoms in women with endometriosis depends. The side effects associated with the lack of estrogens include headache, skin flushing, profuse sweating and atrophic vaginitis. The androgenic side effects include acne, edema, hirsutism, increased weight, and changes to the vocal timber. Better results are obtained after using intrauterine devices with danazole.

GnRH agonist analogues are pharmacological agents which inhibit hypophyseal secretion of gonadotropins by down-regulation of GnRH receptors, and induce a state of hypogonadotropic hypogonadism similar to the menopausal state, which reduces the dimensions and activity of the endometrial implants. Just as danazole, they are counter-indicated during pregnancy and have hypoestrogenism-related side effects. It has, in particular, been shown that they may induce a loss of bone mass. Other effects are: atypical blood loss, hot flushes, vaginal dryness, diminished libido, mammary swelling, insomnia and irritability.

Oral contraceptives suppress LH and FSH and prevent ovulation. Furthermore, they act directly on the endometrium, making it thin and compact. They are mainly administered in non-serious cases for variable periods of time or until the woman expresses the wish to become pregnant.

Finally, progestins inhibit the release of GnRH, FSH and LH, inducing anovulation and amenorrhea. Thus, a hypoestrogenic environment is created which is supposed to lead to progressive decidualization and to endometrial atrophy with necrosis and resorption of the ectopic deposits. Side effects are spotting and intermenstrual bleeding, which require an increased dosage or the combination with an estrogen. They can moreover induce water retention, increased body weight, alterations of the lipid profile, depression, and mammary congestion.

It is clearly evident from the above that it is still not possible today to treat, in a manner that is satisfactory and efficient for the patient, post-surgical pain due to endometriosis and, in particular, pain originating from a post-surgical relapse of endometriosis, reducing this to an at least endurable level, or eliminating it altogether.

There is hence still a need for a system which allows the above-described therapeutic result to be obtained.

It is the aim of the present invention to adequately respond to the aforementioned need of reducing or eliminating the painful symptomatology after surgery and/or from a post-surgical relapse of endometriosis.

These and further aims, which will become clearly apparent from the following detailed description, have been attained by the Applicant who has unexpectedly found that the administration of a food composition comprising one or more essential fatty acids is capable of providing the desired response to the problems laid out above.

One aspect of the present invention is thus the use of essential fatty acids for preparing the aforementioned food composition, as laid down in the enclosed independent claim.

Preferred embodiments of the present invention are given in the enclosed dependent claims.

As will be shown in the following detailed description, and in particular in the experimental section, the Applicant has unexpectedly found that an appropriate food composition, which comprises one or more essential fatty acids and is administered for an appropriate period of time to patients having undergone surgery for endometriosis, has proven to be particularly efficient in treating post-surgical pain in that it significantly reduces or even eliminates the latter.

Said composition has, in particular, proven to be equally efficient also in treating pain originating from a post-surgical relapse of endometriosis, significantly reducing or even eliminating this.

Said food composition comprises at least one essential fatty acid. Said composition preferably comprises a mixture of two or more essential fatty acids; more preferably, said composition comprises a mixture of two said acids.

Said essential fatty acids are preferably selected from the group consisting of long-chain polyunsaturated fatty acids of the omega-3 series.

Preferably, said essential fatty acids of the omega-3 series are selected from the group comprising: eicosapentaenoic acid (EPA) and docosahexaenoic acid (DHA).

In a preferred embodiment of the invention, said food composition comprises an efficient amount of EPA. Said amount preferably ranges from 100 mg to 1000 mg.

In another preferred embodiment of the invention, said food composition comprises an efficient amount of DHA. Said amount preferably ranges from 80 mg to 300 mg.

In a particularly preferred embodiment, said food composition comprises a mixture of EPA and DHA. The total amount of said mixture preferably ranges from 150 mg to 1300 mg (EPA+DHA). In said mixture, EPA+DHA are preferably present in a reciprocal weight ratio from 5:1 to 1:5; said weight ratio of EPA:DHA preferably is about 3.5:1.

The food composition according to the invention is preferably formulated as a mixture with appropriate excipients such as carriers, lubricants, dispersing agents, flavoring agents, sweeteners, stabilizers, preservatives, antioxidants, additives, such as, for example, amino acids, vitamins and enzymes commonly used in formulation technology in the food additive sector.

Only as examples that are absolutely not limiting can amide, tween, aromas such as those of tangerine, grapefruit, strawberry, blueberry, mixed fruits, saccharose, glucose, acesulfame, saccharin, aspartame, ascorbic acid, parabens, glutamine, arginine, superoxide dismutase, glutathione, glycerol, liposoluble vitamins, dyes and soy lecithin be cited among the particularly preferred excipients and additives.

Particularly preferred compositions of the present invention are those for oral and/or topical administration.

Typical preferred forms of formulation are, for example, capsules, drops, ready-to-drink solutions, compresses, bars, emulsions, creams and gels.

The compositions of the present invention are prepared in a traditional manner using, depending on the type of formulation that is to be produced, preparation techniques that are known to those skilled in this field.

Fatty acids of the omega-3 series are normally present in marine food (mainly in salmon and mackerel which, in addition, are also optimal DHA sources) and in some plants. The most commonly occurring omega-3 fatty acid in the plant world is alpha-linolenic acid (LNA). This fatty acid must be transformed into EPA and DHA, respectively, in order to exert the biological effects that are decisive, as is now known, for the proper functioning of some organs and systems, such as the brain, retina and gonads, and which, in addition, offer protection against atherosclerosis and cardio-vascular diseases. EPA and DHA are the most important long-chain fatty acids of the ω-3 series and fulfill structural and functional functions in the human organism.

DHA predominantly has a structural function; in fact, it is mostly present in the phospholipids of brain synaptosomes, in the retina and the phospholipids of intramembrane sodium channels. It thus plays an important role in the development and maturation of the brain, the reproductive apparatus and the retinal tissue. Furthermore, DHA is a precursor of important molecules with antiinflammatory action, i.e. the resolvins of the D series.

EPA is the main precursor of 3-series prostaglandins, which have a potent anti-platelet-aggregation activity. The biological activity of ω-3 fatty acids (antiatherogenic, antiinflammatory, antithrombotic) depends on the prevalence of protective factors over risk factors. It is known, for example, that the intake of ω-3 fatty acids increases the formation of prostaglandin PGI3, the production of leukotrienes B5, of interleukin-2, of EDRF and of E-series resolvins.

In the following examples, two of the food compositions that have proven to be particularly preferred for the use as claimed by the present invention will now be illustrated by way of example, and by no means as a limitation of the invention.

### EXAMPLE 1

### Composition and Dosage of a Food Product Comprising DHA that is Commercially Known as GESTALYS DHA® (Pharmanutra S.r.l.)

GESTALYS DHA® is a DHA-based food supplement with vitamins and minerals that is formulated in a specific manner in accordance with the RDA (recommended daily allowance) of pregnant women. Said product is used to provide supplemental nutrients particularly useful in this period of a woman's life.

Ingredients:

DHA-rich algal oil; alimentary gelatin; firming agent: glycerol; LTPOFER® (ferric pyrophosphate, corn starch, soy lecithin in liposomes); ferrous fumarate; vitamin C; humidifier: E-475, vitamin B3 (niacin); emulsifier: soy lecithin; zinc oxide; beta carotene 30% (beta carotene, sunflower oil); vitamin E (d-alpha-tocopherol, sunflower oil); vitamin B5 (calcium pantotenate); sodium fluoride; vitamin B12 0.1% (cyanocobalamin); vitamin B6 (pyridoxine chlorohydrate); copper sulfate; vitamin B2 (riboflavin); vitamin B1 (thiamine mononitrate); vitamin D3 (chole-calcifero; coconut oil); folic acid; potassium iodide; sodium selenite pentahydrate; biotin; vitamin K1 (phytomenadione); dyes: E-171, E-172. Average recommended daily allowance based on the active ingredients (per 1 capsule/drop a day):

| | | |
|---|---|---|
| DHA | 200.00 | mg |
| Vitamin C | 70.00 | mg |
| Niacin | 14.00 | mg |
| Beta carotene | 1.80 | mg |
| Vitamin E | 4.00 | mg |
| Vitamin B5 | 3.00 | mg |
| Vitamin B6 | 1.60 | mg |
| Vitamin B12 | 2.00 | mcg |
| Vitamin B2 | 0.80 | mg |
| Vitamin B1 | 0.70 | mg |
| Vitamin D | 10.00 | mcg |
| Folic acid | 400.00 | mcg |
| Biotin | 75.00 | mcg |
| Vitamin K | 35.00 | mcg |
| Iron | 30.00 | mg |
| Zinc | 7.00 | mg |
| Copper | 600.00 | mcg |
| Fluorine | 0.50 | mg |
| Iodine | 150.00 | mcg |
| Selenium | 30.00 | mcg |

### EXAMPLE 2

### Composition and Dosage of a Food Product Comprising EPA and DHA that is Commercially Known as ENDOMERAL® (Pharmanutra S.r.l.)

ENDOMERAL® is an EPA- and DHA-based food supplement used to provide a supplemental amount of such nutrients in cases of dietary deficiencies or increased organic requirements.

Ingredients:

Fish oil (MEG-3, 60% EPA, Ocean Nutrition); alimentary gelatin, DHA-rich algal oil (DHA-S, Martek); firming agent: glycerol; vitamin E; soy derivatives.

Average recommended daily allowance based on the active ingredients (per 2 capsules/drops a day):

| | |
|---|---|
| Proteins | 0.54 g |
| Carbohydrates | 0.25 g |
| Fats, of which: | 1.74 g |
| saturated fatty acids | 0.16 g |
| monounsaturated fatty acids | 0.07 g |
| EPA | 0.70 g |
| DHA | 0.20 g |
| Vitamin E | 0.030 g |

The efficiency of the compositions of the present invention has been demonstrated by clinical studies performed on patients having undergone surgery for endometriosis.

The preliminary results of some of said clinical studies are indicated, purely by way of example and not as a limitation, in the following experimental section.

The aim of said studies was to evaluate both the effectiveness of daily administration of DHA and EPA+DHA for pelvic pain in the follow-up examination of women having undergone an operation for endometriosis, and the possible pregnancy rate.

Criteria for Taking Part:

The study included patients that had been positively diagnosed with pelvic endometriosis by means of laparoscopy and accurate staging of the disease according to the criteria established by the American Fertility Society (AFS). Prior to surgery, the patients were subjected to transvaginal ultrasound, determination of ovarian marker (CA 125) levels, and evaluation of pain in accordance with the visual analogue scale (VAS) (Kruskall-Wallis test).

Materials and Methods:

The patients included in the study were divided into two groups:

Study/Group 1—Women trying to get pregnant:

A—10 women were treated with placebo.

B—10 women were treated with the composition of example 1.

Study/Group 2—Women not trying to get pregnant:

A—10 women were treated with the composition of example 2.

B—10 women were treated with placebo.

Dosage of the administered food supplements:

Study/Group 1B: 1 cps per day (corresponding to 200 mg of DHA)

Study/Group 2A: 2 cps per day (corresponding to 350 mg of EPA+100 mg of DHA per drop)

Duration of treatment: 3 months+3 months of follow-up

Duration of the study: 6 months

Follow-Up

The women were subjected to the following controls:
After THREE MONTHS: checkup+TVS+CA-125 level determination+VAS
After SIX MONTHS: checkup TVS+CA-125 level determination+VAS

Preliminary Results:

The preliminary results of the study/group 1 regarding the group of women who had received the composition of example 1 showed a significant reduction of chronic pelvic pain after the first month of treatment (p<0.001); this reduction was even more pronounced three months after the start of treatment.

Furthermore, the group of women (1 B) who had received the composition of example 1 experienced a statistically significant reduction of pain after both one and three months of treatment as compared to the corresponding group who had been treated with placebo (1 A).

As regards the group of women operated for endometriosis who had received the composition of example 2 (2 A), the data is almost identical to that of the preceding group (1 B), both as regards the significant reduction after one and three months of treatment and as regards the efficiency of the treatment with respect to the group treated with placebo (2 B).

1. Use of essential fatty acids for the preparation of a food composition for treating post-surgical endometriosis pain.

2. The use according to claim 1, wherein the pain originates from a post-surgical relapse of endometriosis.

3. The use according to claim 1, wherein said treatment is directed at reducing the pain.

4. The use according to claim 1, wherein said treatment is directed at eliminating the pain.

5. The use according to claim 1, wherein said essential fatty acids are selected from the group consisting of long-chain polyunsaturated fatty acids of the omega-3 series.

6. The use according to claim 5, wherein said fatty acids of the omega-3 series are selected from the group comprising EPA and/or DHA.

7. The use according to claim 1, wherein said composition comprises at least one, preferably from two to four, of said fatty acids.

8. The use according to claim 7, wherein said composition comprises EPA.

9. The use according to claim 8, wherein EPA is present in said composition in an amount ranging from 100 mg to 1000 mg.

10. The use according to claim 7, wherein said composition comprises DHA.

11. The use according to claim 10, wherein DHA is present in said composition in an amount ranging from 80 mg to 300 mg.

12. The use according to claim 10, wherein DHA is administered at an average daily dose of about 200 mg.

13. The use according to claim 7, wherein said composition comprises a mixture of EPA+DHA.

14. The use according to claim 13, wherein the total amount of said mixture, EPA+DHA, in said composition ranges from 150 mg to 1300 mg.

15. The use according to claim 13, wherein EPA and DHA are present in said composition in a weight ratio of 5:1 to 1:5, and the weight ratio of EPA:DHA is preferably about 3.5:1.

16. The use according to claim 13, wherein the mixture EPA+DHA is administered at an average daily total dose of about 900 mg, wherein the amount of EPA is about 700 mg and the amount of DHA is about 200 mg.
